(19) United States
(12) Patent Application Publication
(10) Pub. No.: US 2022/0257540 A1
(43) Pub. Date: Aug. 18, 2022

(54) COMPOSITIONS AND METHODS FOR TREATMENT OF HEPATITIS B VIRUS INFECTION

(71) Applicant: ANHUI RONGHANG BIOTECH DEVELOPMENT CO., LTD., Hefei (CN)

(72) Inventor: Huiqun Zhang, Hefei (CN)

(21) Appl. No.: 17/622,152

(22) PCT Filed: Jun. 28, 2019
(86) PCT No.: PCT/CN2019/093545

§ 371 Date: Dec. 22, 2021

(51) Int. Cl. A61K 31/165; A61K 31/216; A61K 31/277; A61K 31/5377; A61K 31/7048; A61P 31/00

(52) U.S. Cl. CPC A61K 31/165; A61K 31/216; A61P 31/00; A61K 31/5377; A61K 31/7048; A61K 31/277

(57) ABSTRACT

A pharmaceutical composition and kits thereof for treatment of HBV infection comprises colchicine and CYP3A4/P-gp inhibitor.

### TECHNICAL FIELD

The present disclosure relates, in general, to a combinational use of colchicine or a derivative thereof with a CYP3A4/P-gp inhibitor for treatment of hepatitis B virus (HBV) infection. In particular, the instant disclosure relates to a pharmaceutical composition comprising colchicine or a derivative thereof and a CYP3A4/P-gp inhibitor, a method for treatment of HBV infection using colchicine or a derivative thereof in combination with a CYP3A4/P-gp inhibitor, and a kit comprising colchicine or a derivative thereof and a CYP3A4/P-gp inhibitor.

### BACKGROUND

Colchicine is a plant alkaloid. It is effective against gouty arthritis and other forms of rheumatic diseases, such as rheumatoid arthritis, familial Mediterranean fever, and Bechet's disease. Colchicine inhibits the migration of granulocytes into the inflamed area and inhibits the metabolic and phagocytic activity of granulocytes. Further, colchicine is anti-mitotic and anti-fibrotic. Colchicine retards the microtubule-mediated transport of procollagen and enhances collagenase activity.

In addition, colchicine was found to decrease liver fibrosis in rats with carbon tetrachloride induced cirrhosis. Several randomized clinical trials have addressed the question whether colchicine has any efficacy in patients with alcoholic or non-alcoholic fibrosis and cirrhosis. The results have largely been negative, however, although some studies found efficacy of colchicine on mortality.

Colchicine was proposed to treat HBV infection. A pilot study and subsequently a two-center trial have been carried out to assess the efficacy of colchicine for the treatment of chronic Hepatitis B. In both clinical trials, colchicine was given 1 mg a day orally for 5 days-a-week over 6 months. Results showed colchicine's antiviral activity but at a low response rate (3 of 7 failed in the pilot study; in the two-center trial, a sustained response was reached in 4 of 6 treated patients (66.6%) and in 2 of 6 untreated patients (33.3%)). See Floreani, A, et al. “Colchicine in chronic hepatitis B: a pilot study.” Alimentary Pharmacology & Therapeutics 12.7(1998):653-656 and Floreani, A. “Preliminary results of a two-center trial with colchicine for the treatment of chronic Hepatitis B.” The American Journal of Gastroenterology 96.12(2001):3451-3452. A double-blind controlled trial of colchicine in a much larger cohort of 100 patients with hepatitis B virus-related cirrhosis were reported (Wang, Y. J, et al. “A Double-Blind Randomized Controlled Trial of Colchicine in Patients with Hepatitis B Virus-Related Postnecrotic Cirrhosis.” Journal of Hepatology 21.5(1994):872-7). The report indicated that colchicine has no effect in the treatment of HBV-related postnecrotic cirrhosis. The report further discussed that for HBV-related cirrhosis, where viral replication is the major determining factor, the progression of the disease may be inevitable unless there was early treatment with antiviral or antifibrotic drugs.

Hepatitis B virus (HBV) is a non-cytopathic hepadnavirus that chronically infects more than 350 million people worldwide. Chronic hepatitis B virus (HBV) infection is due to the failure of a host to mount a sufficient immune response to clear the virus. The outcomes and pathogenesis of HBV infection are largely determined by the nature and magnitude of host antiviral immune response, which is generally related to the age at the time of infection. While over 95% of adult-acquired infections are spontaneously cleared within 6 months by a vigorous and polyclonal HBV-specific T cell response, more than 90% of exposed neonates and approximately 30% of children aged 1-5 years develop chronic infection, which is associated with a weaker and often barely detectable viral specific T cell response.

The genome of HBV is made of circular DNA, but it is unusual because the DNA is not fully double-stranded. One end of the full-length strand is linked to the viral DNA polymerase. The genome is 3020-3320 nucleotides long (for the full-length strand) and 1700-2800 nucleotides long (for the shorter strand). The negative-sense (non-coding) is complementary to the viral mRNA. The viral DNA is found in the nucleus soon after infection of the cell. There are four known genes encoded by the genome, called C, X, P, and S. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The HBsAg gene is one long open reading frame but contains three in frame “start” (ATG) codons that divide the gene into three sections, pre-S1, pre-S2, and S. Because of the multiple start codons, polypeptides of three different sizes called large, middle, and small are produced. The function of the protein coded for by gene X is not fully understood but it is associated with the development of liver cancer. Replication of HBV is a complex process. Although replication takes place in the liver, the virus spreads to the blood where viral proteins and antibodies against them are found in infected people.

Hepatitis B virus particle (sometimes referred to as a virion) includes an outer lipid envelope and an icosahedral nucleocapsid core composed of protein. The nucleocapsid encloses the viral DNA and a DNA polymerase that has reverse transcriptase activity. The outer envelope contains embedded proteins that are involved in viral binding of, and entry into, susceptible cells, typically liver hepatocytes. In addition to the infectious viral particles, filamentous and spherical bodies lacking a core can be found in the serum of infected individuals. These particles are not infectious and are composed of the lipid and protein that forms part of the surface of the virion, and is produced in excess during the life cycle of the virus.

Sustained suppression of viral replication with long-term nucleos(t)ide analogue therapy or through a finite-duration of pegylated alpha interferon (IFN-α) therapy has been associated with improvement of liver diseases, prevention of liver decompensation and reduction of hepatocellular carcinoma morbidity and mortality. However, HBV surface antigen (HBsAg) seroconversion, the hallmark of a successful immunologic response to HBV with complete and durable control of infection, or a “functional cure,” is rarely achieved with the current therapies.

Acute hepatitis B infection does not usually require treatment and most adults clear the infection spontaneously. Early antiviral treatment may be required in fewer than 1% of people, whose infection takes a very aggressive course (fulminant hepatitis) or who are immunocompromised. On the other hand, treatment of chronic infection may be necessary to reduce the risk of cirrhosis and liver cancer. Chronically infected individuals with persistently elevated serum alanine aminotransferase, a marker of liver damage, and HBV DNA levels are candidates for therapy. Treatment lasts from six months to a year, depending on medication and genotype. Treatment duration when medication is taken by mouth, however, is more variable and usually longer than one year.

Although none of the available medications can clear the infection, they can stop the virus from replicating, thus minimizing liver damage. As of 2018, there are eight medications licensed for the treatment of hepatitis B infection in the United States. These include antiviral medications lamivudine, adefovir, tenofovir disoproxil, tenofovir alafenamide, telbivudine, and entecavir, and the two immune system modulators interferon alpha-2a and PEGylated interferon alpha-2a. In 2015 the World Health Organization recommended tenofovir or entecavir as first-line agents. Those with current cirrhosis are in most need of treatment.

### SUMMARY OF THE INVENTION

The present disclosure provides a pharmaceutical composition, comprising an amount of colchicine or a pharmaceutically acceptable salt thereof, and a pharmacokinetically effective amount of a CYP3A4/P-gp inhibitor. In some embodiments, the CYP3A4/P-gp inhibitor exists in an amount to increase a peak concentration (Cmax) of colchicine by about 30% to about 500%. In some embodiments, the CYP3A4/P-gp inhibitor exists in an amount to increase a peak concentration (Cmax) of colchicine by about 50% to about 200%.

In some embodiments, the amount of colchicine is between about 0.6 mg and about 2.0 mg. In some embodiments, the amount of colchicine is between about 0.6 mg and about 1.2 mg. In some embodiments, the amount of colchicine is between about 1.0 mg.

In some embodiments, the CYP3A4/P-gp inhibitor is selected from a group consisting of ritonavir, clarithromycin, 1-aminobenzotriazole, proadifen, chloramphenicol, ketoconazole, itraconazole, cobicistat, cyclosporine and verapamil. In some embodiments, the CYP3A4/P-gp inhibitor is ritonavir, 1-aminobenzotriazole, proadifen, or cobicistat.

In some embodiments, the pharmaceutical composition is administered to obtain a peak concentration (Cmax) of colchicine between about 20 nM and about 60 nM. In some embodiments, the pharmaceutical composition is administered to obtain a peak concentration (Cmax) of colchicine between about 30 nM and about 40 nM.

The present disclosure also provides a kit for treatment of hepatitis B virus infection in a subject comprising a first pharmaceutical composition comprising an amount of colchicine, and a second pharmaceutical composition comprising a pharmacokinetically effective amount of a CYP3A4/P-gp inhibitor.

A further aspect of this disclosure provides a method for treatment of HBV infection in a subject comprising administering to the subject in need thereof an amount of colchicine and a pharmacokinetically effective amount of a CYP3A4/P-gp inhibitor, such that the peak concentration (Cmax) of colchicine in the subject is intentionally increased to a predetermined level. In some embodiments, the Cmax of colchicine is increased by about 30% to about 500%.

A further aspect of this disclosure provides a method for enhancing an anti-HBV efficacy of colchicine in a subject for treatment of HBV infection, comprising co-administering to the subject a pharmacokinetically effective amount of a CYP3A4/P-gp inhibitor, such that the peak concentration (Cmax) of colchicine in the subject is intentionally increased to a pre-determined level. In some embodiments, the Cmax of colchicine in the subject is increased by about 30% to about 500%. In some embodiments, the Cmax of colchicine in the subject is increased to about 20 nM to about 60 nM.

Other aspects or advantages of the disclosure will be apparently obtainable by a skilled person in the art from the following detailed description of the invention.

### BRIEF DESCRIPTION OF THE DRAWINGS

FIG. 1. In vitro efficacy study of colchicine in HBV-infected PXB-cells. Shown were representative microscopic images of HBV-infected PXB cells untreated (A1/A2) and treated with different concentrations of colchicine (B1/B2: 31.3 nM, C1/C2: 125 nM, D1/D2: 1000 nM). Upper: magnification 40×, Lower: magnification 100×).

FIG. 2. Body weight changes of in vivo experiments during treatment and off-treatment period. G01: vehicle, 10 ml/kg, PO, QD, Day 0-41, n=4; G02: colchicine, 1/2/5 mg/kg, PO, QD, Day 0-41, n=4; G03: colchicine+ritonavir, 1/2/5 mg/kg, PO, QD, Day 0-41, n=4. PO=per oral, QD=quaque die (once a day).

FIG. 3. Serum viral marker levels during treatment and off-treatment periods. (A) Serum HBsAg, (B) Serum HBV-DNA, *P<0.05, **P<0.01 vs. G01, (C) Serum HBeAg, *P<0.05 vs. G01. G01: vehicle, 10 ml/kg, PO, QD, Day 0-41, n=4; G02: colchicine, 1/2/5 mg/kg, PO, QD, Day 0-41, n=4; G03: colchicine+ritonavir, 1/2/5 mg/kg, PO, QD, Day 0-41, n=4. PO=per oral, QD=quaque die (once a day).

FIG. 4. Serum ALT and AST during treatment and off-treatment periods. (A) Serum ALT, (B) Serum AST. G01: vehicle, 10 ml/kg, PO, QD, Day 0-27, n=4; G02: colchicine, 1/2/5 mg/kg, PO, QD, Day 0-27, n=4; G03: colchicine+ritonavir, 1/2/5 mg/kg, PO, QD, Day 0-27, n=4. PO=per oral, QD=quaque die (once a day).

FIG. 5. In vivo experiments using AAV-HBV model to check efficacy of different treatment. Control: vehicle, 10 ml/kg, PO, QD, n=3; colchicine, 3.3 mg/kg, PO, QD, n=3; colchicine+Cobicistat, PO, QD, n=3; or colchicine+1-Aminobenzotriazole PO, QD, n=3 for 7 days. Then the viral markers were measured, *P<0.05, **P<0.01, ***P<0.001.

FIG. 6. Body weight changes of mice administered with colchicine and colchicine plus various CYP3A4/P-gp inhibitors at indicated dosages. colchicine, 5 mg/kg (n=5); Colchicine+Cobicistat, 5 mg/kg+5 mg/kg (n=5); Colchicine+Proadifen, 5 mg/kg+10 mg/kg (n=5); Colchicine+Ritonavir, 5 mg/kg+10 mg/kg (n=5); Colchicine+1-Aminobenzotriazole(1-ABT), 5 mg/kg+50 mg/kg (n=5).

### DETAILED DESCRIPTION OF THE INVENTION

### Definitions

It is to be noted that the term “a” or “an” entity refers to one or more of that entity; for example, “a pharmaceutically acceptable salt” is understood to represent one or more pharmaceutically acceptable salts. As such, the terms “a” (or “an”), “one or more,” and “at least one” can be used interchangeably herein.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. In the present invention, the term “treat” or “treatment” also refer to the reduce or eliminate of the the surface antigen (HBsAg), the core antigen (HBcAg) and/or the e antigen (HBeAg), or the reduce or eliminate of Hepatitis B virus DNA amount, as detected by blood sample tests commonly used in the art. In the present invention, the term “treat” or “treatment” also refer to HBeAg seroconversion in the subject treated according to the present invention.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on. The subject herein is preferably a human.

As used herein, phrases such as “to a subject in need thereof” or “a subject in need of treatment” includes subjects, such as mammalian subjects, that would benefit from administration of colchicine or a composition of the present disclosure used, e.g., for detection, for a diagnostic procedure and/or for treatment.

As used herein, the term “infection” means the invasion by, multiplication and/or presence of a pathogen in a cell, tissue, or subject. In one embodiment, an infection is an “active” infection, i.e., one in which the pathogen is replicating in a cell, tissue, or subject. Such an infection may be characterized by the spread of the pathogen to other cells, tissues, organs, and/or subjects from the cells, tissues, organs, and/or subjects initially infected by the pathogen. An infection may also be a latent infection, i.e., one in which the pathogen is not replicating. In one embodiment, an infection refers to the pathological state resulting from the presence of the pathogen in a cell, tissue, or subject, or by the invasion of a cell, tissue, or subject by the pathogen.

As used herein, the term “pharmaceutically acceptable salts” refers to derivatives of the colchicine wherein the it is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from colchicine by conventional chemical methods. Generally, such salts can be prepared by reacting the free acid or base forms of the compound with a stoichiometric amount of the appropriate base or acid in water or in an organic solvent, or in a mixture of the two; generally, nonaqueous media like ether, ethyl acetate, ethanol, isopropanol, or acetonitrile are preferred. A non-limiting example is colchicine salicylate.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. Other additional ingredients that may be included in the pharmaceutical compositions used in the practice of the invention are known in the art.

Colchicine and Derivatives

The chemical name of colchicine is N [5,6,7,9-tetrahydro-1,2,3,10-tetratmethoxy 9-oxobenzo[a]heptalen-7-yl], (S)-acetamide. It exists in two forms (−)-(aS,7S)-colchicine and (+)-(aR,7S)-colchicine, which interconvert quickly when the compound is in solution and at ambient temperatures. The ratio of the two conformers is 99:1. The molecular formula is C₂₂H₂₅NO₆ and the molecular weight is 399.4 g/mole. Colchicine has a structure of:

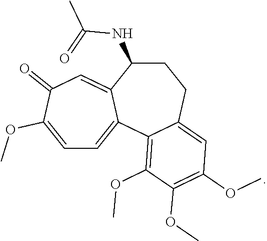

In healthy adults, colchicine capsules when given orally reached a mean Cmax of 3 ng/mL in 1.3 h (range 0.7 to 2.5 h) after 0.6 mg single dose administration, or 6.50±1.03 ng/mL in 1.07±0.55 hours after 1.0 mg single dose administration. For prophylaxis of gout flares, the recommended dosage of colchicine capsules is 0.6 mg once or twice daily. The maximum dose is 1.2 mg per day. Absolute bioavailability is reported to be approximately 45%.

The pharmacokinetic profiles of different regimens of colchicine have been obtained in healthy volunteers who had fasted. Low-dose colchicine (1.2 mg followed by 0.6 mg in 1 h; 1.8 mg total), high-dose colchicine (1.2 mg followed by 0.6 mg every hour for 6 h; 4.8 mg total) and single dose (0.6 mg) colchicine were given to n=13, n=15 and n=25 volunteers, respectively. Pharmacokinetic sampling occurred over 96 h. Peak blood levels (Cmax) for single-dose, low-dose and high-dose colchicine were 2.5, 6.19 and 6.84 ng/ml, respectively. Exposure to colchicine (area under the curve) was 14.1, 52.1 and 118.2 ng/h/ml, respectively. The terminals half-lives for single-dose, low-dose and high dose colchicine were 6.36, 23.6 and 31.4 h, respectively (Terkeltaub R A et al. High versus low dosing of oral colchicine for early acute gout flare: twenty-four-hour outcome of the first multicenter, randomized, double-blind, placebo-controlled, parallel-group, dose-comparison colchicine study. Arthritis Rheum 2010; 62:1060-8). These data indicate that low- and high-dose colchicine regimens achieved a comparable peak blood colchicine concentration of ˜6 ng/ml in normal, healthy volunteers.

Colchicine has a narrow therapeutic window and is extremely toxic in overdose. Patients at particular risk of toxicity are those with renal or hepatic impairment, gastrointestinal or cardiac disease, and patients at extremes of age. A review of 150 patients who overdosed on colchicine found that those who ingested less than 0.5 mg/kg survived and tended to have milder adverse reactions, such as gastrointestinal symptoms, whereas those who ingested from 0.5 to 0.8 mg/kg had more severe adverse reactions, including myelosuppression. There was 100% mortality among patients who ingested more than 0.8 mg/kg.

Colchicine derivatives are known in the art. Exemplary derivatives include N-[(7S)-1,2,3-trimethoxy-9-oxo-10-[3-(trifluoromethyl)-4-chlorophenylamino]-5,6,7,9-tetrahydrobenzo[a]heptalen-7-yl]acetamide, 4-halocolchicines, thiocolchicine and any of those described in Majcher, U. et al. Antiproliferative Activity and Molecular Docking of Novel Double-Modified Colchicine Derivatives. Cells 2018, 7, 192 and Baljinder Singh et al. Colchicine derivatives with potent anticancer activity and reduced P-glycoprotein induction liability. Org. Biomol. Chem., 2015, 13, 5674-5689. Other derivatives are also available. Techniques of chemistry modification are available to a skilled person in the art. The present disclosure contemplates that colchicine derivatives are usable with a CYP3A4/P-gp inhibitor to achieve same or similar results as described herein and thus is within the scope of the disclosure.

CYP3A4/P-gp Inhibitors

The cytochrome P450 (CYP) is a superfamily of enzymes that are responsible for the oxidative and reductive metabolic transformation of medications used in clinical practice. Of the CYP enzymes, CYP3A4, localizes to the endoplasmic reticulum, and its expression is induced by glucocorticoids and some pharmacological agents. The human cytochrome P450 enzyme system present in the liver and intestine is responsible for the metabolism of a wide range of xenobiotics (for example drugs, carcinogens and pesticides) and endobiotics (for example prostaglandins, bile acids and steroids). CYP3A4 is responsible for the metabolism (at least in part) of more than 50% of marketed drugs. It is the ability of CYP3A4 to metabolize numerous structurally unrelated compounds that makes CYP3A4 responsible for the poor oral bioavailability of many drugs as they are subjected to pre-systemic CYP3A4-mediated metabolic activity. Pre-systemic metabolism of pharmaceutical compounds occurs when orally administered pharmaceutical compounds are metabolized during their passage to the systemic circulation from the gut lumen. Typical organs that play an important role in pre-systemic metabolism include, for example the liver and the intestine where CYP3A4-mediated metabolic activity is known to occur.

P-glycoprotein (P-gp) is a 170-kDa glycoprotein encoded by the MDR1 (ABCB1) gene located on chromosome 7q21.12 and was first identified in the CHO cell line. P-gp is a member of the ATP-binding cassette (ABC) transporter family and is capable of energy dependent transport of a variety of intracellular substrates. P-gp is located within the plasma membrane and functions to extrude xenobiotic agents against their concentration gradient. P-gp is constitutively expressed on multiple cell types including the apical membrane of intestinal mucosal cells, the brush border of renal proximal tubules, the blood-brain barrier, and lower airway epithelial cells. Due to the selective distribution at the port of drug entry and exit, P-gp functions as a biochemical barrier for entry of xenobiotics and as a vacuum cleaner to expel them from the organs, such as brain, liver, kidney, and ultimately from systemic circulation. This xenobiotic excretion function belies the role of P-gp in reducing the systemic bioavailability of a variety of drugs.

The term “CYP3A4/P-gp inhibitor(s)”, “inhibitor(s)” as used herein refers to a substance that has inhibitory effect on the activity of the CYP3A4 enzyme, the P-glycoprotein transporter, or both. Therefore, the meaning of the term “CYP3A4/P-gp inhibitor(s)” includes an CYP3A4 inhibitor, a P-gp inhibitor, and a dual inhibitor of CYP3A4 and P-gp (i.e, a substance that acts both as a CYP3A4 inhibitor and a P-gp inhibitor, for example itraconazole and clarithromycin).

Colchicine is a substrate of the efflux transporter P-glycoprotein (P-gp), and the CYP3A4 metabolizing enzyme. Fatal drug interactions have been reported when colchicine is administered with clarithromycin, a dual inhibitor of CYP3A4 and P-glycoprotein. Toxicities have also been reported when colchicine is administered with inhibitors of CYP3A4 that may not be potent inhibitors of P-gp (e.g., grapefruit juice, erythromycin, verapamil), or inhibitors of P-gp that may not be potent inhibitors of CYP3A4 (e.g., cyclosporine).

Patients with renal or hepatic impairment was not recommended to be given colchicine capsules with drugs that inhibit both P-glycoprotein and CYP3A4. Combining these dual inhibitors with colchicine capsules in patients with renal and hepatic impairment has resulted in life-threatening or fatal colchicine toxicity.

The term “CYP3A4 inhibitor” as used herein refers to an inhibitor of cytochrome P450 3A4 (CYP3A4). It is contemplated that any known and pharmacologically acceptable compound capable of inhibiting cytochrome P450, i.e. inhibiting CYP3A4 isoenzyme, is useful in the present invention, unless otherwise specified.

Examples of useful CYP3A4 inhibitors are ritonavir, indinavir, nelfinavir, saquinavir, clarithromycin, grapefruit juice, 1-Aminobenzotriazole, telithromycin, chloramphenicol, ketoconazole, itraconazole, posaconazole, voriconazole, nefazodone, cobicistat, amiodarone, aprepitant, verapamil, erythromycin, fluconazole, miconazole, bergamottin, cimetidine, ciprofloxacin, cyclosporine, donedarone, fluvoxamine, imatinib, valerian, verapamil, buprenorphine, cafestol, cilostazol, fosaprepitant, gabapentin, lomitapide, orphenadrine, ranitidine, ranolazine, tacrolimus, ticagrelor, valproic acid, cannabidiol, dithiocarbamate, mifepristone, norfloxacin, delavirdine, gestodene, mibefradil, star fruit, milk thistle, niacinamide, Ginkgo biloba, piperine, isoniazid, quercetin, lanzoprazol, safrole, rutaecarpine, limonin, dipiperamide A, gomisin C, paradisin A and paradisin B. In some embodiment, the present invention excludes the use of diltiazem from the CYP3A4 inhibitor.

Some of the CYP3A4 inhibitors are protease inhibitors which include, for example, amprenavir, fosamprenavir, atazanavir, darunavir, indinavir, lopinavir, nelfinavir, ritonavir, saquinavir, tipranavir and combinations thereof. In a particular example, the CYP3A4 inhibitor comprises ritonavir.

A CYP3A4 inhibitor can be classified as a strong, moderate, or weak inhibitor based on its effect on colchicine. A strong inhibitor increases the AUC of a sensitive index CYP substrate ≥5-fold. A moderate inhibitor increases the AUC of a sensitive index CYP substrate by ≥2- to <5-fold. A weak inhibitor increases the AUC of a sensitive index CYP substrate by ≥1.25- to <2-fold. The present invention contemplates use of any of a strong, moderate and weak CYP3A4 inhibitor.

A number of inhibitors of P-gp are known in the art (Varma et al., Pharmacological Research 2003; 48: 347-359). In general, P-gp can be inhibited (1) by blocking its substrate binding site; (2) by interfering with its ATPase activity; or (3) by decreasing its expression level either transcriptionally or posttranscriptionally. Based on specificity and affinity, P-gp inhibitors are classified into three generations.

First-generation P-gp inhibitors are known pharmacological compounds that are in clinical use, or were developed for, for other indications but have been shown to inhibit P-gp. These include calcium channel blockers such as verapamil; immunosuppressants like cyclosporin A; anti-hypertensives, reserpine, quinidine and yohimbine; and anti-estrogens like tamoxifen and toremifena. The usage of these compounds has been limited by their toxicity due to the high serum concentrations achieved with the dose that is required to inhibit P-gp when administered systemically.

Second-generation P-gp modulators are agents that lack the pharmacological activity of the first-generation compounds and usually possess a higher P-gp affinity. Second-generation P-gp inhibitors include the non-immunosuppresive analogues of cyclosporin A such as PSC 833 (Valspodar: 6-[(2S,4R,6E)-4-methyl-2-(methylamino)-3-oxo-6-octenoic acid]-7-L-valine-cyclosporin A); verapamil isomers such as D-isomer of verapamil, R-verapamil, and dexverapamil; and other inhibitors such as VX-710 (Biricodar: 1,7-di(pyridin-3-yl)heptan-4-yl (2S)-1-[oxo(3,4,5-trimethoxyphenyl)acetyl]piperidine-2-carboxylate); GF 120918 (Elacridar: N-(4-(2-(6,7-dimethoxy-3,4-dihydroisoquinolin-2(1H)-yl)ethyl)phenyl)-5-me-thoxy-9-oxo-9,10-dihydroacridine-4-carboxamide hydrochloride); and MS-209 (Dofequidar fumarate: 1-(4-(2-hydroxy-3-(quinolin-5-yloxy)propyl)piperazin-1-yl)-2,2-diphenylet-hanone).

The third-generation P-gp blockers are under development with the primary purpose to improve the treatment of multidrug resistant tumors and to inhibit P-gp with high specificity and toxicity. Examples of the third-generation P-gp inhibitors include LY335979 (Zosuquidar: (2R)-1-{4-[(1aR,6r,10bS)-1,1-Difluoro-1,1a,6,10b-tetrahydrodibenzo[a,e]cy-clopropa[c]cyclohepten-6-yl]piperazin-1-yl}-3-(quinolin-5-yloxy)propan-2-o-1,trihydrochloride); OC144093 (4-[2-[4-[(E)-3-ethoxyprop-1-enyl]phenyl]-4-[4-(propan-2-ylamino)phenyl]-1H-imidazol-5-yl]-N-propan-2-ylaniline); R-101933 (Laniquidar: methyl 11-(1-(4-(quinolin-2-ylmethoxy)phenethyl)piperidin-4-ylidene)-6,11-dihydr-o-5H-benzo[d]imidazo[1,2-a]azepine-3-carboxylate); XR9576 (Tariquidar: N-[2-[[4-[2-(6,7-Dimethoxy-3,4-dihydro-1H-isoquinolin-2-yl)ethyl]phenyl]c-arbamoyl]-4,5-dimethoxyphenyl]quinoline-3-carboxamide); XR9051 (3-((Z)-((Z)-5-benzylidene-4-methyl-3,6-dioxopiperazin-2-ylidene)methyl)-N-(4-(2-(6,7-dimethoxy-3,4-dihydroisoquinolin-2(1H)-yl)ethyl)phenyl)benza-mide). Some third-generation P-gp modulators such as LY335979, OC144093, and XR9576 are shown to be highly potent and selective inhibitors of P-gp with a potency of about 10-fold more than the first and second-generation inhibitors.

In some embodiments, the P-gp inhibitor is selected from a group consisting of, clarithromycin, itraconazole, ketoconazole, quinidine, verapamil, bepridil, nicardipine, nifedipine, felodipine, isradipine, trifluorperazine, clopenthixol, trifluopromazine, flupenthixol, chlorpromazine, prochlorperazine, indole alkaloids, the anti-malarial quinine, the anti-arrhythmic quinidine, Cyclosporin-A, dexverapamil, emopamil, gallopamil, and Ro11-2933, PSC 833, zosuquidar, elacridar, XR9051, OC144-093, biricodar (VX-710), timcodar (VX-853) or tariquidar (XR9576).

A full list of P-gp inhibitors was described by Hossam M et al, P-glycoprotein inhibitors of natural origin as potential tumor chemo-sensitizers: A review, Journal of Advanced Research, Volume 6, Issue 1, 2015, Pages 45-62.

A skilled person in the art knows how to determine if a substance is an inhibitor of metabolizing enzymes, for example CYP3A4 enzyme. A skilled person in the art also knows how to determine if a substance is an inhibitor of a transporter, for example P-glycoprotein. The reader is directed to In Vitro Metabolism-and Transporter-Mediated Drug-Drug Interaction Studies Guidance for Industry issued in October 2017 by U.S. Department of Health and Human Services, Food and Drug Administration and Center for Drug Evaluation and Research (CDER).

Hepatitis B Virus Infection (HBV Infection)

Hepatitis B is an infection of the liver caused by the hepatitis B virus (HBV). Markers found in the blood can confirm hepatitis B infection and differentiate acute from chronic infection. These markers are substances produced by the hepatitis B virus (antigens) and antibodies produced by the immune system to fight the virus. Hepatitis B virus has three antigens for which there are commonly-used tests: the surface antigen (HBsAg), the core antigen (HBcAg) and the e antigen (HBeAg).

HBsAg and anti-HBs: The presence of hepatitis B surface antigen (HBsAg) in the blood indicates that the patient is currently infected with the virus. HBsAg appears an average of four weeks after initial exposure to the virus. Individuals who recover from acute hepatitis B infections clear the blood of HBsAg within approximately four months after the onset of symptoms. These individuals develop antibodies to HBsAg (anti-HBs). Anti-HBs provides complete immunity to subsequent hepatitis B viral infection. Similarly, individuals who are successfully vaccinated against hepatitis B produce anti-HBs in the blood. Patients who fail to clear the virus during an acute episode develop chronic hepatitis B. The diagnosis of chronic hepatitis B is made when the HBsAg is present in the blood for at least six months. In chronic hepatitis B, HBsAg can be detected for many years, and anti-HBs does not appear.

Anti-HBc: In acute hepatitis, a specific class of early antibodies (IgM) appears that is directed against the hepatitis B core antigen (anti-HBc IgM). Later, another class of antibody, anti-HBc IgG, develops and persists for life, regardless of whether the individual recovers or develops chronic infection. Only anti-HBc IgM can be used to diagnose an acute hepatitis B infection. HBeAg, anti-HBe, and pre-core mutations. Hepatitis B e antigen (HBeAg) is present when the hepatitis B virus is actively multiplying, whereas the production of the antibody, anti-HBe, (also called HBeAg seroconversion) signifies a more inactive state of the virus and a lower risk of transmission. In some individuals infected with hepatitis B virus, the genetic material for the virus has undergone a structural change, called a pre-core mutation. This mutation results in an inability of the hepatitis B virus to produce HBeAg, even though the virus is actively reproducing. This means that even though no HBeAg is detected in the blood of people with the mutation, the hepatitis B virus is still active in these people and they can infect others.

Hepatitis B virus DNA: The best marker of hepatitis B virus reproduction is the level of hepatitis B virus DNA in the blood. Detection of hepatitis B virus DNA in a blood sample signals that the virus is actively multiplying. In acute hepatitis, HBV DNA is present soon after infection, but is eliminated over time in patients' who clear the infection. In chronic hepatitis, levels of HBV DNA often continue to be elevated for many years and then decrease as the immune system controls the virus. HBV DNA levels are sometimes referred to as the ‘viral load’.

Methods and Therapies

The present disclosure provides a method for treatment of HBV infection using colchicine in combination with a CYP3A4/P-gp inhibitor. The use of colchicine alone for treatment of HBV infection, as described above, appears not promising in clinic and further clinical trials were not carried out, resulting in no NDA application or FDA approval.

The present inventors surprisingly found that by boosting the blood concentration of colchicine in a subject with an inhibitor of CYP3A4/P-gp, the efficacy of colchicine is significantly improved in terms of virus clearance and reproduction. The data shown herein indicates that when colchicine is intentionally used in combination with a CYP3A4/P-gp inhibitor, the HBV DNA level (extracellular or intracellular) is significantly reduced compared to the use of colchicine alone.

A CYP3A4/P-gp inhibitor was described to avoid coadministration with colchicine due to drug-drug interaction (DDI) and was reported to result in life-threatening condition or even death. In the case colchicine is administered with a CYP3A4/P-gp inhibitor, e.g. ritonavir, it was proposed to reduce the dose of colchicine either by reducing the intended daily dosage of colchicine or by extending dosing intervals, in order to prevent from potential toxicity. However, in the present disclosure, a CYP3A4/P-gp inhibitor is used in combination with colchicine in an intentional way based on the findings that colchicine is extremely effective to inhibit HBV replication and promote virus clearance when the blood concentration of colchicine is increased to a certain level that is not reachable by a normally recommended dosage of colchicine, for example not more than 2 mg. In some embodiments of the present disclosure, considering the potential toxicity and narrow therapeutic window of colchicine and the normally recommended dosage of colchicine as now practiced in clinic, the increase of blood concentration of colchicine is obtained by co-administering a CYP3A4/P-gp inhibitor to the same subject receiving colchicine dosing without increase of the normal dosage of colchicine.

In some embodiments, the CYP3A4/P-gp inhibitor is co-administered with colchicine such that the peak concentration (Cmax) of colchicine in the blood of the subject receiving the treatment is increased to a level that is effective to reduce HBV replication or clear HBV. The dosage of the CYP3A4/P-gp inhibitor is an amount that pharmacokinetically increases the Cmax of colchicine co-administered with the CYP3A4/P-gp inhibitor in the subject. In the present disclosure, the dosage of the CYP3A4/P-gp inhibitor co-administered with colchicine is defined as “pharmacokinetically effective amount”. The term “pharmacokinetically effective amount” as used in this specification refers to an amount of each active ingredient that can pharmacokinetically increase the Cmax of the colchicine in the subject treated to a desired or predetermined level. By controlling the amount of the inhibitor alone or the dosages of the colchicine and the inhibitor, the increasement of the Cmax in the subject can be adjusted. The pharmacokinetically effective amount of the CYP3A4 inhibitor, the P-gp inhibitor or the dual CYP3A4/P-gp inhibitor for a single dose may be prescribed in a variety of ways, depending on factors such as formulation methods, administration manners, age of patients, body weight, gender, pathologic conditions, diets, administration time, administration interval, administration route, excretion speed, and reaction sensitivity. For example, the pharmacokinetically effective amount of the inhibitor for a single dose may be in ranges of 0.001 to 100 mg/kg, or 0.02 to 10 mg/kg, but not limited thereto. The pharmacokinetically effective amount for the single dose may be formulated into a single formulation in a unit dosage form or formulated in suitably divided dosage forms, or it may be manufactured to be contained in a multiple dosage container. In one embodiment, the effective amount of the inhibitor is about 0.1 mg/kg to about 100 mg/kg. In another embodiment, the effective amount of the inhibitor is about 1 mg/kg to about 20 mg/kg. In one embodiment, the effective amount of the inhibitor (e.g., ritonavir) is about 0.1 mg/kg to about 100 mg/kg. In another embodiment, the effective amount of the inhibitor is about 1 mg/kg to about 20 mg/kg.

In some embodiments, the Cmax of colchicine in the subject is increased by about 30% to about 500%. Preferably, about 50% to about 500%, about 50% to about 400%, about 50% to about 300%, about 50% to about 200%, about 50% to about 180%, about 50% to about 150%, about 50% to about 120%, about 50% to about 100%, about 60% to about 500%, about 60% to about 400%, about 60% to about 300%, about 60% to about 200%, about 60% to about 100%, about 70% to about 500%, about 70% to about 400%, about 70% to about 300%, about 70% to about 200%, about 70% to about 100%, about 80% to about 500%, about 80% to about 400%, about 80% to about 300%, about 80% to about 200%, about 80% to about 100%, about 90% to about 500%, about 90% to about 400%, about 90% to about 300%, about 90% to about 200%, about 90% to about 100%, about 100% to about 500%, about 100% to about 400%, about 100% to about 300%, about 100% to about 200%, about 100% to about 180%, about 100% to about 150%, about 100% to about 120%, or any in between. For example, the Cmax of colchicine in the subject is increased by about 30%, about 40%, about 50%, about 60%, about 70%, about 80%, about 90%, about 100%, about 110%, about 120%, about 130%, about 140%, about 150%, about 160%, about 170%, about 180%, about 90%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500% or any value in between.

The level that is effective to reduce HBV replication or clear HBV, as described above, is determined by pharmacological parameters of colchicine on a subject. In some embodiments, the Cmax of colchicine in the subject is increased to about 20 nM to about 60 nM, preferably about 20 nM to about 55 nM, about 20 nM to about 50 nM, about 20 nM to about 45 nM, about 20 nM to about 40 nM, about 20 nM to about 35 nM, about 20 nM to about 30 nM, about 25 nM to about 60 nM, about 25 nM to about 55 nM, about 25 nM to about 50 nM, about 25 nM to about 45 nM, about 25 nM to about 40 nM, about 25 nM to about 35 nM, about 25 nM to about 30 nM, about 30 nM to about 60 nM, about 30 nM to about 55 nM, about 30 nM to about 50 nM, about 30 nM to about 45 nM, about 30 nM to about 40 nM, about 30 nM to about 35 nM, about 35 nM to about 60 nM, about 35 nM to about 55 nM, about 35 nM to about 50 nM, about 35 nM to about 45 nM, about 35 nM to about 40 nM, about 40 nM to about 60 nM, about 40 nM to about 55 nM, about 40 nM to about 50 nM, about 40 nM to about 45 nM. For example, the Cmax of colchicine in the subject is increased to about 20 nM, about 22 nM, about 24 nM, about 26 nM, about 28 nM, about 30 nM, about 32 nM, about 34 nM, about 36 nM, about 38 nM, about 40 nM, about 42 nM, about 44 nM, about 46 nM, about 48 nM, about 50 nM, about 52 nM, about 54 nM, about 56 nM, about 58 nM, about 60 nM, or any value in between.

In some embodiments, the colchicine is administered at a dosage of about 0.6 mg to about 2 mg once daily, preferably about 0.6 mg to about 1.2 mg once daily, more preferably about 1.0 mg once daily. In some embodiments, the colchicine is administered at a dosage less than about 2 mg daily.

In some embodiments, the inhibitor is administered at a dosage of about 50 mg to about 600 mg once daily, preferably about 100 mg to about 600 mg once daily, preferably about 100 mg to about 500 mg once daily, preferably about 100 mg to about 400 mg once daily, preferably about 100 mg to about 300 mg once daily, preferably about 100 mg to about 200 mg once daily, preferably about 200 mg to about 500 mg once daily, preferably about 200 mg to about 400 mg once daily, preferably about 200 mg to about 300 mg once daily, preferably about 300 mg to about 500 mg once daily, preferably about 400 mg to about 500 mg once daily or any value in between.

The term “co-administering” or similar expressions used herein include scenarios that the colchicine is administered before, simultaneously or after the administering of the CYP3A4/P-gp inhibitor. Preferably the colchicine is administered simultaneously with the inhibitor.

In cases when the colchicine is not administered simultaneously with the inhibitor, it is contemplated that one may administer the subject with both modalities within about 0.1 to 12 hrs of each other. In some embodiments, both modalities are administered within about 0.5 to 6 hrs of each other. In some embodiments, both modalities are administered within about 1 to 6 hrs of each other. In some embodiments, both modalities are administered within about 3 to 6 hrs of each other. For example, the colchicine is administered about 0.1, about 0.2, about 0.4, about 0.6, about 0.8, about 1.0, about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0, about 5.2, about 5.4, about 5.6, about 5.8, about 6.0, or any value in between, before or after the administration of the inhibitor. In some embodiments, the inhibitor is administered before the administration of the colchicine or its derivative. For example, the inhibitor is administered about 0.1, about 0.2, about 0.4, about 0.6, about 0.8, about 1.0, about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0, about 5.2, about 5.4, about 5.6, about 5.8, about 6.0, or any value in between, before the administration of colchicine or its derivative.

In some embodiments, the colchicine and the inhibitor are administered in the form of a pharmaceutical composition comprising an amount of colchicine, a pharmacokinetically effective amount of the inhibitor and a pharmaceutically acceptable carrier. In some embodiments, the colchicine is administered in the form of a pharmaceutical composition comprising an amount of colchicine and a pharmaceutically acceptable carrier, and the inhibitor is administered in the form of a pharmaceutical composition comprising a pharmacokinetically effective amount of inhibitor and a pharmaceutically acceptable carrier. In those embodiments, a kit is provided to include the pharmaceutical composition comprising an amount of colchicine and a pharmaceutically acceptable carrier and the pharmaceutical composition comprising a pharmacokinetically effective amount of inhibitor and a pharmaceutically acceptable carrier. In some embodiments, the kit may contain an instruction for use that a physician can refer to during clinical use.

In certain embodiments, either of the pharmaceutical compositions described above is administered parenterally or non-parenterally, e.g. orally, intravenously, intramuscularly, percutaneously or intracutaneously. Preferably, the pharmaceutical compositions described above is administered orally.

Therefore, an aspect of the disclosure provides a method for treatment of HBV infection in a subject comprising administering to the subject in need thereof an amount of colchicine and a pharmacokinetically effective amount of a CYP3A4/P-gp inhibitor, such that the peak concentration (Cmax) of colchicine in the subject is intentionally increased to a predetermined level. In some embodiments, the Cmax of colchicine is increased by about 30% to about 500%.

In some embodiments, the Cmax of colchicine is increased to a level that is effective to reduce HBV replication or clear HBV. In some embodiments, the extracellular or intracellular HBV DNA level is significantly reduced compared to the use of same amount of colchicine alone.

In some embodiments, the Cmax of colchicine is increased to a level that results in the level of HBsAg and/or HBeAg in the subject be significantly reduced compared to the use of same amount of colchicine alone.

The term “significantly” as used herein means the level or value is increased or decreased by at least about 20% compared to controls (for example, a level or value obtained by same amount of colchicine alone). In some embodiments, “significantly” means a variation by at least about 30%, 40%, 50%, 60%, 70%, 80%, 90%, 100%, 200%, 300%, 400%, 500%, 600%, 700%, 800%, 900%, 1,000%, 5,000%, 10,000% or more, or any value in between.

In some embodiments, the Cmax of colchicine is increased to about 20 nM to about 60 nM, preferably to about 30 nM to about 40 nM.

In some embodiments, the method of the disclosure further comprises monitoring the subject for potential toxicity. In some embodiments, the method of the disclosure further comprises adjusting the dose of colchicine and the inhibitor to avoid adverse side effects.

Another aspect of the disclosure provides a method for enhancing an anti-HBV efficacy of colchicine in a subject for treatment of HBV infection, comprising co-administering to the subject a pharmacokinetically effective amount of a CYP3A4/P-gp inhibitor, such that the peak concentration (Cmax) of colchicine in the subject is intentionally increased to a pre-determined level. In some embodiments, the Cmax of colchicine in the subject is increased by about 30% to about 500%. In some embodiments, the Cmax of colchicine in the subject is increased to about 20 nM to about 60 nM.

Pharmaceutical Compositions and Kits

Another aspect of the present invention provides a pharmaceutical composition comprising an amount of colchicine or a pharmaceutically acceptable salt thereof, a pharmacokinetically effective amount of a CYP3A4/P-gp inhibitor, and a pharmaceutically acceptable carrier. The pharmaceutical composition is useful for treatment of HBV infection in a subject. In some embodiment, the pharmaceutical composition comprises about 0.6 mg to about 2 mg of colchicine.

A further aspect of the present invention provides a kit comprising a first pharmaceutical composition comprising an amount of colchicine or a pharmaceutically acceptable salt thereof, and a pharmaceutically acceptable carrier; and a second pharmaceutical composition comprising a pharmacokinetically effective amount of a CYP3A4/P-gp inhibitor and a pharmaceutically acceptable carrier. In some embodiments, the kit may contain an instruction for use that a physician can refer to during clinical use. In some embodiment, the first pharmaceutical composition comprises about 0.6 mg to about 2 mg of colchicine.

Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 mL of isotonic NaCl solution and either added to 1,000 mL of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

### EXPERIMENTAL EXAMPLES

### Example 1. Colchicine Alone Showed Anti-HBV Efficacy in a Dose Dependent Manner

Materials & Methods: PXB-cells was infected with HBV GT C. Colchicine was treated starting on Day 12 and the treatment continued every 3 days until Day 24 with the end of incubation on Day 27. The anti-HBV therapeutic efficacy was measured by the analysis of extracellular HBV DNA, HBsAg, HBeAg, h-Alb, and intracellular HBV DNA/cceDNA levels. Entecavir was used as the positive control. The hepatocytes isolated from 3 mice were combined to seed culture plate. The cell density of mixed cell was 7.37×10⁶ cells/mL. PXB-cells® are the fresh hepatocytes which are isolated from PXB-mice. Cells viability after isolation: >80%. Donor Hepatocytes were purchased from Corning, Incorporated (Tewksbury, Mass., USA), Lot: BD195 (Hispanic, 2Y, Female). Colchicine was obtained from RIM bioscience LLC, Lot: 20180320, Conc.: 10 mM. Entecavir was purchased from Santa-cruz, Lot: 10914, Concentration: 1 mM. Plates was observed under microscopy and photographed.

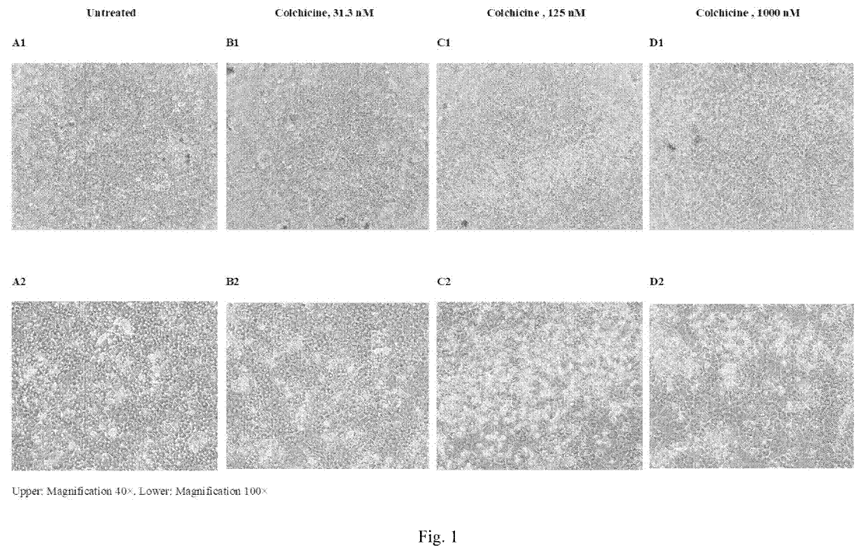

Results & Conclusions: The results of measurements of parameters on Day 27 was given in Table 1. Colchicine and positive control Entecavir both show anti-HBV efficacy in a dose dependent manner. The efficacy of colchicine is robust, though slightly weaker than Entecavir. The microscopic images shown in FIG. 1 demonstrated the does-dependent anti-HBV efficacy of colchicine compared to the untreated control group.

### Example 2. In Vivo Pharmacodynamics Efficacy of Colchicine in AAV-HBV Mouse Model

Materials & Methods

Purpose & Materials: The purpose of this study was to evaluate in vivo pharmacodynamics efficacy of test compounds in AAV-HBV mouse model. Recombinant AAVs carrying the HBV genome rAAV8-1.3HBV ayw (Lot #: 2016120601) was purchased by Covance from Beijing FivePlus Molecular Medicine Institute., and stored at −70° C. before use. Vehicle, Colchicine and Combination of colchicine and Ritonavir were provided as ready to use solution and stored at 2˜8° C. before dosing.

Animals and Housing: Sixteen male C₅₇BL/6 mice aged 4˜5 weeks were received on 18 Sep. 2017 from Shanghai Lingchang Bio Tech Co. Ltd, Shanghai, China. The mice were selected for inclusion in the study based upon acceptable clinical condition and body weight. Mice were group housed in polycarbonate cages with corncob bedding under controlled temperature (21-25° C.), humidity (40-70%), and a 12-hour light/12-hour dark cycle (7:00 AM to 7:00 PM lights on). Mice were provided ad libitum access to normal diet (Rodent Diet #5CC4, PMI Nutrition International, LLC, IN, USA) and sterile water. All procedures in this study were in compliance with local animal welfare legislation, Covance global policies and procedures, and the Guide for the Care and Use of Laboratory Animals.

Mouse AAV-HBV Model Induction: The animals were acclimatized in the animal facility for 7 days after arrival at Covance. On Day −28, all the animals were subjected to injection through tail vein (IV) with 1×10¹¹ vector genome of AAV-HBV (in 200 μL, PBS) for model induction. Animal clinical signs were monitored daily and body weights were measured on Days −35, −28, −14 and −7. On Days −14 and −7 (14 and 21 days post AAV-HBV injection, respectively), blood samples were collected via retro-orbital sinus bleeding under anesthesia by isoflurane for serum preparation (15 μL per mouse), and determined for baseline viral load analysis.

Treatment Parameters: On Day −1 (one day before the 1st compound dosing), based on serum viral markers (HBsAg, HBV DNA, HBeAg) and body weight on Day −7, 12 qualified HBV-infected mice were selected and randomized into 3 groups, with 4 animals per group, for compound treatment (Table 2).

TABLE 1
Measurements of Parameters on Day 27

| | | | | Extracellular | | | | Intracellular | | |
|---|---|---|---|---|---|---|---|---|---|---|
| Group | | | | HBV DNA | HBsAg | HBeAg** | hAlb | HBV DNA | cccDNA | Total DNA |
| No. | Substances | Concentration | | (Log copies/mL) | (IU/mL) | (C.O.I) | (μg/mL) | (copies/100 ng DNA) | (copies/100 ng DNA) | (ng/well) |
| 1 | Control | — | | 7.16 | 28.5 | 18.9 | 82.3 | 321974 | 325 | 15295 |
| 2 | Colchicine | 1 | nM | 7.06 | 23.1 | 14.7 | 85.3 | 250511 | 217 | 16135 |
| 3 | | 2 | nM | 7.06 | 23.5 | 16.2 | 89.3 | 235131 | 207 | 16310 |
| 4 | | 3.9 | nM | 7.16 | 26.7 | 19.2 | 85.7 | 282895 | 247 | 15695 |
| 5 | | 7.8 | nM | 7.06 | 25.5 | 17.4 | 84.6 | 270324 | 227 | 14875 |
| 6 | | 15.6 | nM | 6.86 | 25.2 | 16.3 | 71.2 | 256994 | 261 | 14960 |
| 7 | | 31.3 | nM | 6.06 | 2.17 | 3.6 | 20.7 | 52249 | 80 | 13390 |
| 8 | | 62.5 | nM | 4.86 | 0.12 | 0.7 | <5.0 | 9638 | + | 10980 |
| 9 | | 125 | nM | 4.86 | <0.05 | 0.3 | <5.0 | 8341 | + | 8985 |
| 10 | | 250 | nM | 4.86 | <0.05 | 0.2 | <5.0 | 9187 | + | 8165 |
| 11 | | 500 | nM | 4.86 | <0.05 | 0.2 | <5.0 | 9019 | + | 8550 |
| 12 | | 1000 | nM | 4.86 | <0.05 | 0.2 | <5.0 | 8717 | 29 | 8395 |
| 13 | Control | — | | 7.06 | 26.7 | 18.9 | 91.3 | 247161 | 230 | 16595 |
| 14 | Entecavir | 9.8 | pM | 6.36 | 16.0 | 11.6 | 90.9 | 91546 | 125 | 17125 |
| 15 | | 19.5 | pM | 5.76 | 14.2 | 10.0 | 91.2 | 53766 | 82 | 17250 |
| 16 | | 39.1 | pM | 5.06 | 12.2 | 8.1 | 92.8 | 30701 | 69 | 17515 |
| 17 | | 78.1 | pM | 4.66 | 15.7 | 9.8 | 92.1 | 14257 | 82 | 17305 |
| 18 | | 156.3 | pM | 4.56 | 15.1 | 9.6 | 92.2 | 5620 | 61 | 17200 |
| 19 | | 312.5 | pM | 4.46 | 14.3 | 9.4 | 94.6 | 3752 | 34 | 16440 |
| 20 | | 625 | pM | 4.46 | 13.0 | 8.9 | 95.2 | 2954 | + | 16710 |
| 21 | | 1.25 | nM | 4.46 | 11.2 | 6.9 | 92.0 | 3031 | + | 17430 |
| 22 | | 2.5 | nM | 4.46 | 10.8 | 7.3 | 94.1 | 2484 | + | 17130 |
| 23 | | 5 | nM | 4.26 | 10.9 | 7.1 | 93.7 | 2974 | 33 | 15775 |
| 24 | | 10 | nM | 4.46 | 11.6 | 7.4 | 86.2 | 2936 | + | 16880 |

Limit of quantification: HBV-DNA (extracellular); 2.76 Log copies/ml, HBsAg; 0.05 IU/mL, HBeAg; Cut off index, hAlb; 5.0 μg/mL, HBV DNA (intracellular); 10 copies/100 ng DNA, cccDNA; 25 copies/100 ng DNA.
*: These values have not been confirmed by quality control yet.
**The HBeAg level was measured by using 10 fold diluted sample.
+: Real time PCR positive.

Vehicle was dosed at 10 mL/kg during Day 0˜17 and Day 23˜41, and at 20 mL/kg during Day 18˜22. Colchicine and Colchicine+Ritonavir were dosed at 1 mg/kg by 10 mL/kg during Day 0˜17, at 2 mg/kg by 20 mL/kg during Day 18˜22, at 5 mg/kg by 10 mL/kg during Day 23˜41. Vehicle and both compounds were dosed once daily by oral gavage. Animal clinical signs were monitored daily. Body weights were measured twice weekly for dosing calculation during treatment period and once weekly during off-treatment period. Blood samples were collected once weekly (prior to dosing during treatment period) from all mice for serum preparation (30 μL per mouse) on Days 0, 7, 14, 21, 28, 35, 42, 49 and 56. The serum samples were used for the measurement of HBsAg, HBeAg and HBV DNA by Adicon, and measurement of AST and ALT by Covance. On Day 56, after blood collection, all animals on study were euthanized without tissue collection. The four unqualified HBV-infected mice not enrolled in study were euthanized without blood or tissue collection. Based on the serum HBV biomarkers (HBV DNA, HBsAg and HBeAg) and body weight on Day −7, animals were sorted into 3 groups. Compound treatment was initiated on Day 0 in animals with average HBsAg of 4.28 Log 10 IU/mL, HBV DNA of 5.72 Log 10 IU/mL, HBeAg of 3.66 Log 10 S/CO and average body weight of 23.2 g. The combinational uses of colchicine with Cobicistat and with 1-Aminobenzotriazole follow same procedure as identified above with reference to Ritonavir.

TABLE 2
Group design for compound treatment

| | | | | Dose | | | |
|---|---|---|---|---|---|---|---|
| | | No. of | Dosage | Volume | Dose | Dose | Dose |
| Group | Treatment | Animals | (mg/kg) | (mL/kg) | Route | Frequency | Duration |
| 01 | Vehicle | 4 | 0 | 10/20 | PO | QD | Day 0~41 |
| 02 | Colchicine | 4 | 1/2/5 | 10/20 | PO | QD | Day 0~41 |
| 03 | Colchicine + Ritonavir | 4 | 1/2/5 | 10/20 | PO | QD | Day 0~41 |

Abbreviations: PO = Oral dosing; QD =Once daily.

TABLE 3
Composition of test compounds

| | | Dosage | Colchicine | Ritonavir |
|---|---|---|---|---|
| Group | Treatment | (mg/kg) | (mg) | (mg) |
| 02 | Colchicine | 1/2/5 | 1/2/5 | 0 |
| 03 | Colchicine + Ritonavir | 1 | 1 | 1.5 |
| 03 | Colchicine + Ritonavir | 2 | 2 | 3 |
| 03 | Colchicine + Ritonavir | 5 | 5 | 7.5 |

Results and Conclusion

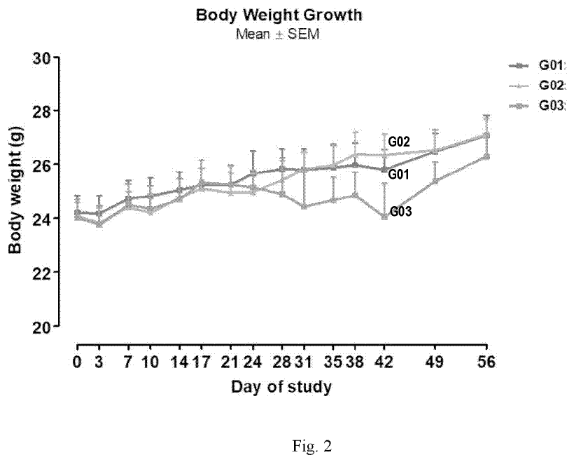

As shown in FIG. 2, no significant difference in body weight growth was observed among the study groups, except slight decrease in Group 03 (G03) during Day 21-42 while the dose level increased.

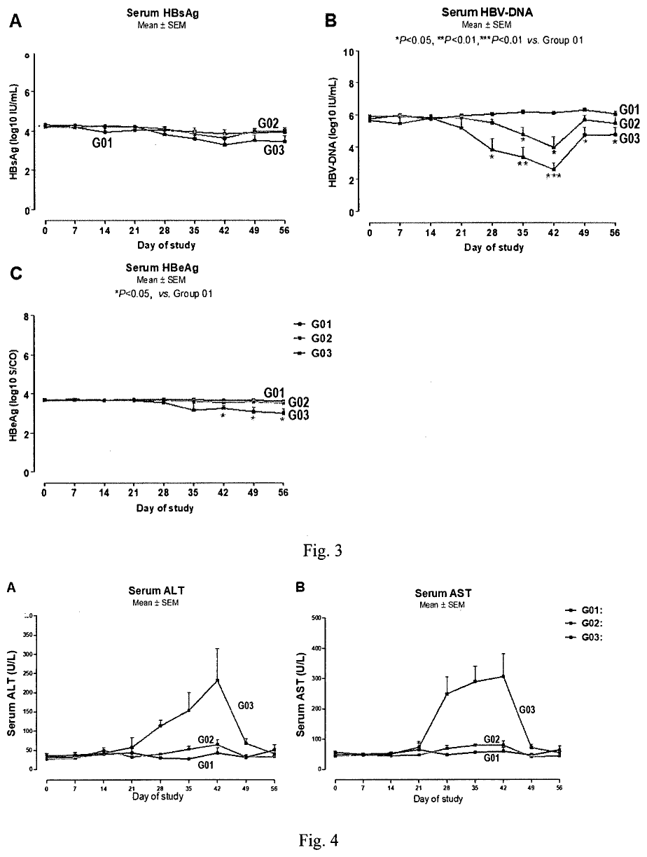

When the dose level was increased to 2 mg/kg/day and 5 mg/kg/day, Colchicine and Colchicine+Ritonavir showed significant downregulating effect on serum HBV-DNA, which rebounded after treatment cessation since Day 42 (FIG. 3). The serum levels of HBV-DNA and HBeAg in Colchicine+Ritonavir treated group kept significantly lower than vehicle group during the off-treatment period (Day 42-56).

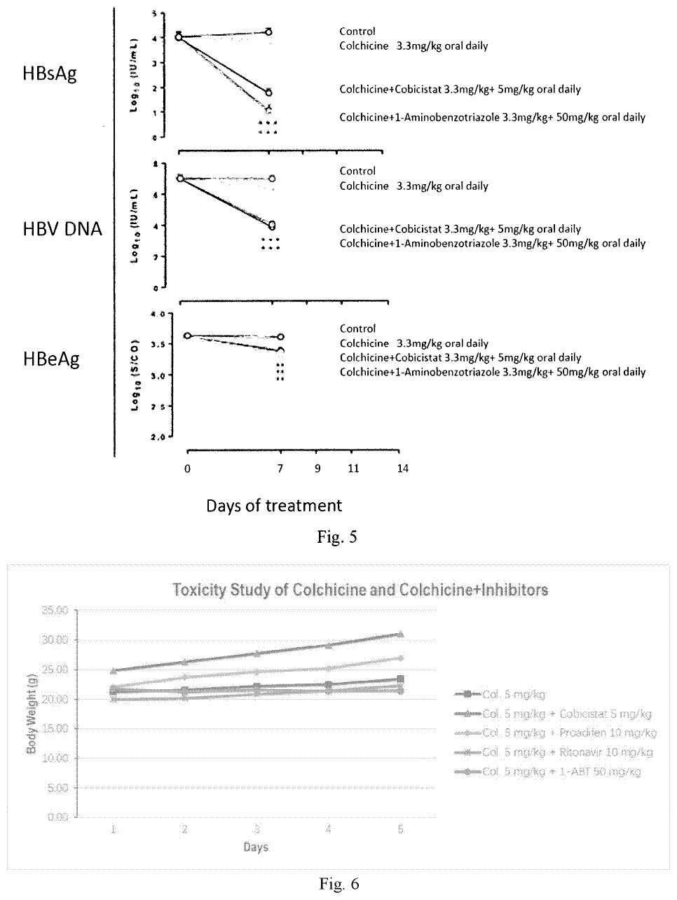

As shown in FIG. 4, the serum ALT and AST in Colchicine+Ritonavir treated group increased since Day 28 along with the dose level increment, and recovered to normal level after treatment cessation. FIG. 5 shows the in vivo efficacy of colchicine with another inhibitors, which indicates that combinational uses of colchicine with cobicistat, and colchicine with 1-Aminobenzotriazole significantly reduce the levels of HBsAg, HBV DNA and HBeAg, compared to colchicine alone, in AAV-HBV mouse model.

### Example 3. Toxicity Studies of Colchicine in Mice

Materials and Methods: Male BalB/c mice (18-21 g) approximately 4-5 weeks old were used for the assay. The animals were divided randomly into five groups as below. Oral administered indicated concentrations of Colchicine or Colchicine plus indicated various CYP3A4/P-gp inhibitors once daily. The mouse weights are measured and recorded daily. If animals have weight loss no more than 15% or the body weight was over 15 g, all mice received another oral administration. When the mice died or and weight loss was more than 15%, the experiment was ended. Mice were divided into 5 groups at random: (Oral once daily) Control (Colchicine) (n=5); Colchicine+Cobicistat (n=5); Colchicine+Proadifen (n=5); Colchicine+Ritonavir (n=5); Colchicine+1-Aminobenzotriazole(1-ABT) (n=5).

Results were shown in FIG. 6 in which the change of body weights of mice appear not significant among groups. Combinational use of Colchicine with different CYP3A4/P-gp inhibitors as indicated was not more toxic to mice compared to colchicine alone. In particular, when the dosage of one of the inhibitors (1-ABT) increased to 50 mg/kg, the body weights of mice did not reduce significantly, indicating good tolerances to the combination.

It should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

1. A pharmaceutical composition for the treatment of hepatitis B virus infection, comprising an amount of colchicine or a derivative thereof, and a pharmacokinetically effective amount of a CYP3A4/P-gp inhibitor.

2. The pharmaceutical composition of claim 1, wherein the CYP3A4/P-gp inhibitor exists in an amount to increase a peak concentration (Cmax) of colchicine or a derivative thereof by about 30% to about 500%.

3. The pharmaceutical composition of claim 1, wherein the CYP3A4/P-gp inhibitor exists in an amount to increase a peak concentration (Cmax) of colchicine or a derivative thereof by about 50% to about 200%.

4. The pharmaceutical composition of claim 1, wherein the amount of colchicine or a derivative thereof is between about 0.6 mg and about 2.0 mg.

5. The pharmaceutical composition of claim 1, wherein the amount of colchicine or a derivative thereof is between about 0.6 mg and about 1.2 mg.

6. The pharmaceutical composition of claim 1, wherein the amount of colchicine or a derivative thereof is between about 1.0 mg.

7. The pharmaceutical composition of claim 1, wherein the CYP3A4/P-gp inhibitor is selected from a group consisting of ritonavir, clarithromycin, 1-aminobenzotriazole, proadifen, chloramphenicol, ketoconazole, itraconazole, cobicistat, cyclosporine and verapamil.

8. The pharmaceutical composition of claim 7, wherein the CYP3A4/P-gp inhibitor is ritonavir, 1-aminobenzotriazole, proadifen, or cobicistat.

9. The pharmaceutical composition of claim 1, wherein the pharmaceutical composition is administered to obtain a peak concentration (Cmax) of colchicine or a derivative thereof between about 20 nM and about 60 nM.

10. The pharmaceutical composition of claim 1, wherein the pharmaceutical composition is administered to obtain a peak concentration (Cmax) of colchicine or a derivative thereof between about 30 nM and about 40 nM.

11. The pharmaceutical composition of claim 1, wherein the derivative of colchicine is N-[(7S)-1,2,3-trimethoxy-9-oxo-10-[3-(trifluoromethyl)-4-chlorophenylamino]-5,6,7,9-tetrahydrobenzoheptalen-7-yl]acetamide, 4-halocolchicines or thiocolchicine.

12. A kit for treatment of hepatitis B virus infection in a subject comprising a first pharmaceutical composition comprising an amount of colchicine or a derivative thereof, and a second pharmaceutical composition comprising a pharmacokinetically effective amount of a CYP3A4/P-gp inhibitor.

13. The kit of claim 12, wherein the derivative of colchicine is N-[(7S)-1,2,3-trimethoxy-9-oxo-10-[3-(trifluoromethyl)-4-chlorophenylamino]-5,6,7,9-tetrahydrobenzo[a]heptalen-7-yl]acetamide, 4-halocolchicines or thiocolchicine.

14. The kit of claim 12, wherein the CYP3A4/P-gp inhibitor is selected from a group consisting of ritonavir, clarithromycin, 1-aminobenzotriazole, proadifen, chloramphenicol, ketoconazole, itraconazole, cobicistat, cyclosporine and verapamil.

15. The kit of claim 12, wherein the CYP3A4/P-gp inhibitor is ritonavir, 1-aminobenzotriazole, proadifen, or cobicistat.

16. The kit of claim 12, wherein the pharmacokinetically effective amount increases a peak concentration (Cmax) of colchicine or a derivative thereof by about 30% to about 500%.

17. The kit of claim 12, wherein the pharmacokinetically effective amount increases a peak concentration (Cmax) of colchicine or a derivative thereof by about 50% to about 200%.

18. The kit of claim 12, wherein the pharmacokinetically effective amount obtains a peak concentration (Cmax) of colchicine or a derivative thereof between about 20 nM and about 60 nM.

19. The kit of claim 12, wherein the pharmacokinetically effective amount obtains a peak concentration (Cmax) of colchicine or a derivative thereof between about 30 nM and about 40 nM.
